(19) United States
(12) Patent Application Publication
(10) Pub. No.: US 2021/0068840 A1
(43) Pub. Date: Mar. 11, 2021

(54) ENERGIZABLE SURGICAL CLIP APPLIER

(71) Applicant: Covidien LP, Mansfield, MA (US)

(72) Inventors: James D. Allen, IV, Broomfield, CO (US); Tyler J. Bagrosky, Arvada, CO (US)

(73) Assignee: Covidien LP, Mansfield, MA (US)

(21) Appl. No.: 17/012,576

(22) Filed: Sep. 4, 2020

Related U.S. Application Data
(60) Provisional application No. 62/896,743, filed on Sep. 6, 2019.

(51) Int. Cl. A61B 17/128; A61B 17/29; A61B 18/12; A61B 18/14

(52) U.S. Cl. CPC A61B 17/1285; A61B 17/2909; A61B 2018/126; A61B 18/1442; A61B 2018/1253; A61B 18/1206

(57) ABSTRACT

A clip applier includes a handle, an elongated shaft, an end effector, at least one surgical clip, a trigger, and an actuation mechanism. The trigger is configured to selectively translate at least a portion of the first jaw member relative to the second jaw member upon actuation thereof in a direction parallel to the longitudinal axis. The actuation mechanism is adapted to connect to a source of electrosurgical energy and is configured to selectively transmit energy to at least one of the at least one surgical clip.

### CROSS REFERENCE TO RELATED APPLICATIONS

This application claims the benefit of U.S. Provisional Application Ser. No. 62/896,743, filed Sep. 6, 2019, the entire contents of which are incorporated by reference herein.

### BACKGROUND

### Technical Field

The disclosure relates to surgical instruments such as, for example, surgical clip appliers. More particularly, the disclosure relates to surgical clip appliers capable of supplying energy, e.g. monopolar energy, to one or more surgical clip(s) for use as energy-delivery electrodes, e.g., before deployment of the clip(s), at one or more partially-deployed positions, after deployment of the clip(s), etc. Related methods are also provided.

### Description of Related Art

Surgical clip appliers are used for a number of distinct and useful surgical procedures. Surgical clip appliers having various sizes (e.g., diameters) are configured to apply a variety of diverse surgical clips and are capable of applying single or multiple surgical clips during cavity surgical procedure. Such surgical clips are typically fabricated from a biocompatible material and are usually compressed over tissue. Once applied to tissue, the compressed surgical clip terminates the flow of fluid therethrough.

Monopolar instruments are often used to dissect and/or coagulate vessels by applying energy to the vessel.

### SUMMARY

The disclosure relates to a clip applier. The clip applier includes a handle, an elongated shaft, an end effector, at least one surgical clip, a trigger, and an actuation mechanism. The elongated shaft extends distally from the handle. The end effector is operably engaged to a distal portion of the elongated shaft, includes a longitudinal axis defined therethrough, and includes a first jaw member and a second jaw member. The at least one surgical clip is disposed within the elongated shaft and is selectively loadable between the first and second jaw members. The trigger is configured to selectively translate at least a portion of the first jaw member relative to the second jaw member upon actuation thereof in a direction that is parallel to the longitudinal axis. The actuation mechanism is adapted to connect to a source of electrosurgical energy and is configured to selectively transmit energy to one of the at least one surgical clip.

In disclosed embodiments, a distal portion of the first jaw member is movable relative to the second jaw member and relative to a proximal portion of the first jaw member to deform a surgical clip of the at least one surgical clip.

In aspects of the disclosure, the actuation mechanism is configured to transmit energy to a distal-most surgical clip of the at least one surgical clip. It is disclosed that the transmission of energy occurs during formation of the distal-most surgical clip, while the first jaw member is stationary relative to the second jaw member, while at least a portion of the first jaw member is longitudinally translating relative to the second jaw member via actuation of the trigger, while at least a portion of the first jaw member is distally translating relative to the second jaw member via actuation of the trigger, and/or when the distal-most surgical clip is engaging tissue.

It is further disclosed that actuation of the trigger is configured to cause at least a portion of the first jaw member to move relative to the second jaw member in a direction that is at a non-parallel angle relative to the longitudinal axis.

Additionally, it is disclosed that actuation of the trigger is configured to cause translation of the second jaw member relative to the elongated shaft in a direction that is parallel to the longitudinal axis.

The disclosure also relates to a method of treating tissue. The method includes translating at least a portion of a first jaw member of a clip applier relative to a second jaw member of the clip applier, while simultaneously moving a surgical clip from a first position where the surgical clip is in contact with the first jaw member and the second jaw member, to a second position where the surgical clip engages tissue and dislodges from the first and second jaw members, and energizing the surgical clip.

In aspects of the disclosure, the method also includes automatically moving a second surgical clip from a position proximally of the first position into the first position.

Additionally, embodiments of the method include translating at least a portion of the first jaw member relative to the second jaw member causes formation of the surgical clip.

Further, in embodiments of the method, energizing the surgical clip is performed during formation of the surgical clip, while the first jaw member is stationary relative to the second jaw member, while at least a portion of the first jaw member is longitudinally translating relative to the second jaw member, and/or while the surgical clip is engaging tissue.

Embodiments of the method also include moving at least a portion of the first jaw member relative to the second jaw member in a direction that is non-parallel relative to the longitudinal axis.

### BRIEF DESCRIPTION OF THE DRAWINGS

Aspects and features of the disclosed surgical clip applier are described in detail with reference to the drawing figures wherein like reference numerals identify similar or identical structural elements and:

FIG. 1 is a perspective view of a surgical clip applier in accordance with an embodiment of the disclosure;

FIG. 2 is a perspective view, with parts separated, of an elongated assembly of the surgical clip applier of FIG. 1;

FIG. 3 is a top, plan view of the elongated assembly of FIGS. 1 and 2;

FIG. 4 is an exploded, perspective view of the elongated assembly of FIGS. 1-3

FIGS. 5 and 6 are side views of surgical clips usable with the surgical clip applier of FIGS. 1-4;

FIGS. 7-9 are side views of jaw members of the surgical clip applier of FIGS. 1-4 at different stages of operation and including and illustrating deployment of the surgical clip of FIG. 5;

FIGS. 10-13 are sides views of jaw members of a surgical clip applier according to an embodiment of the disclosure at different stages of operation and illustrating deployment of a surgical clip; and

FIG. 14 is a schematic illustration of a robotic surgical system configured for use in accordance with the disclosure.

### DETAILED DESCRIPTION OF EMBODIMENTS

Embodiments of the surgical clip applier will now be described in detail with reference to the drawing figures wherein like reference numerals identify similar or identical structural elements. As shown in the drawings and described throughout the following description, as is traditional when referring to relative positioning on a surgical instrument, the term “proximal” refers to the end of the apparatus which is closer to the user and the term “distal” refers to the end of the apparatus which is farther away from the user.

As used herein, the term parallel is understood to include relative configurations that are substantially parallel up to about + or −10 degrees from true parallel.

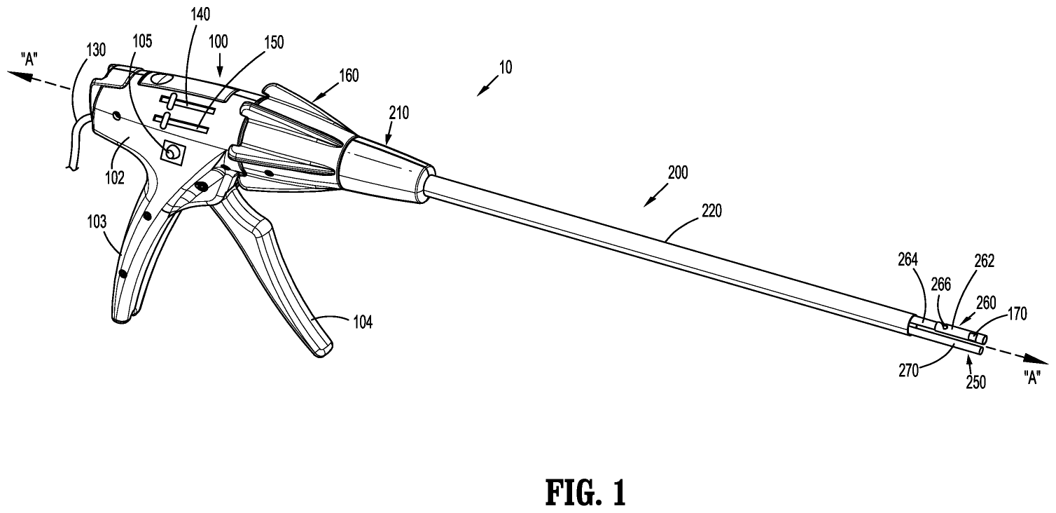

Referring now to FIGS. 1-4, a surgical clip applier in accordance with an embodiment of the disclosure is shown, and is generally designated as reference number 10. Surgical clip applier 10 includes a handle assembly 100, an elongated assembly 200 extending distally from handle assembly 100 and defining a longitudinal axis “A-A,” and a pair of jaw members 250 extending distally from elongated assembly 200.

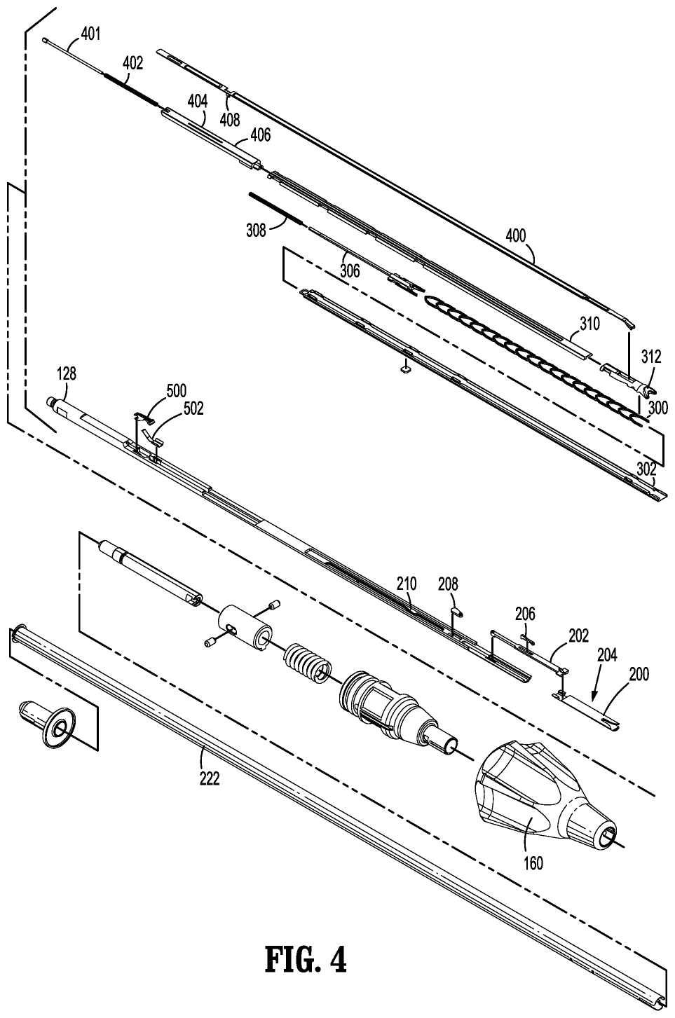

Elongated assembly 200 or a surgical clip cartridge assembly (not shown) may be loaded with a particularly sized set of surgical clips 300 (e.g., relatively small surgical clips, relatively medium surgical clips, or relatively large surgical clips), an example of which is shown in FIG. 4. It is contemplated that clip cartridge assemblies may be configured to be selectively loaded into the shaft assembly of elongated assembly 200, and to be actuated by the same or common handle assembly 100 to deploy and form the surgical clip(s) 300 loaded therein onto underlying tissue and/or vessels.

Handle assembly 100 of surgical clip applier 10 is shown in FIG. 1. Generally, handle assembly 100 includes a housing 102, a stationary handle 103, a trigger 104, a drive plunger (not shown), a first slider 140, a second slider 150, and a rotation knob 160. Drive plunger is operatively connected to trigger 104 and is slidably supported within housing 102 of handle assembly 100. Actuation of trigger 104 toward stationary handle 103 distally advances the drive plunger relative to housing 102. Translation of first slider 140 results in a corresponding translation of a first jaw member 260 of end effector 250, and translation of second slider 150 results in a corresponding translation of a second jaw member 270 of end effector 250. Further, in embodiments, an additional amount of translation of first slider 140 results in the pivoting or camming of a distal portion 262 of first jaw member 260 relative to a proximal portion 264 of first jaw member 260 about pin 266. Rotation knob 160 is disposed at a distal portion of housing 102 and is configured to enable elongated assembly 200 to rotate 360° about a longitudinal axis thereof relative to housing 102 of handle assembly 100.

The elongated or assembly 200 of surgical clip applier 10 is shown in FIGS. 1-3. Elongated assembly 200 includes a hub assembly 210, and a shaft assembly including elongated shaft 220 extending from hub assembly 210.

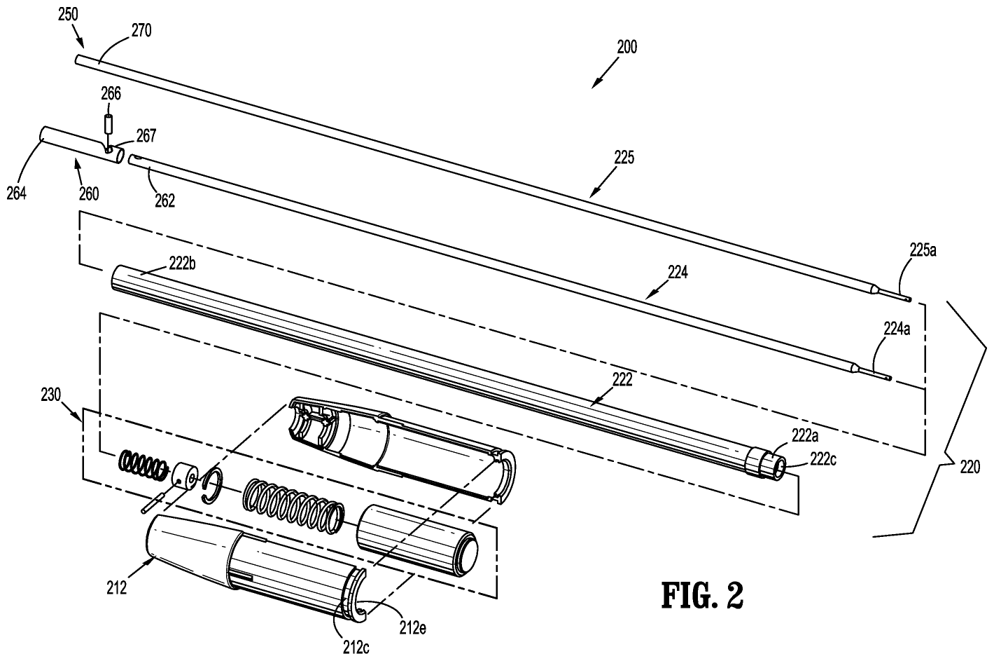

With particular reference to FIG. 2, an outer housing 212 of hub assembly 210 further defines an open proximal end 212e configured to slidably receive a distal end of the drive plunger of handle assembly 100, when elongated assembly 200 is coupled to handle assembly 100 and/or when surgical clip applier 10 is actuated.

Shaft assembly 220 of elongated assembly 200 includes an elongated outer tube 222 having a proximal end 222a supported and secured to outer housing 212 of hub assembly 210, a distal end 222b projecting from outer housing 212 of hub assembly 210, and a lumen 222c (FIG. 2) defined therein and extending longitudinally therethrough. As shown in FIG. 2, hub assembly 210 includes a drive assembly 230 supported within outer housing 212 thereof. Jaw members 250 extend through distal end 222b of outer tube 222.

Shaft assembly 220 further includes first shaft 224 and a second shaft 225 slidably supported within lumen 222c of outer tube 222. First shaft 224 includes a proximal end 224a projecting proximally from proximal end 222a of outer tube 222 and which is disposed in mechanical cooperation with first slide 140 of handle assembly 100. First jaw member 260 is disposed at a distal end of first shaft 224. Second shaft 225 includes a proximal end 225a projecting proximally from proximal end 222a of outer tube 222 and which is disposed in mechanical cooperation with second slide 150 of handle assembly 100. Second jaw member 270 is disposed at a distal end of second shaft 225.

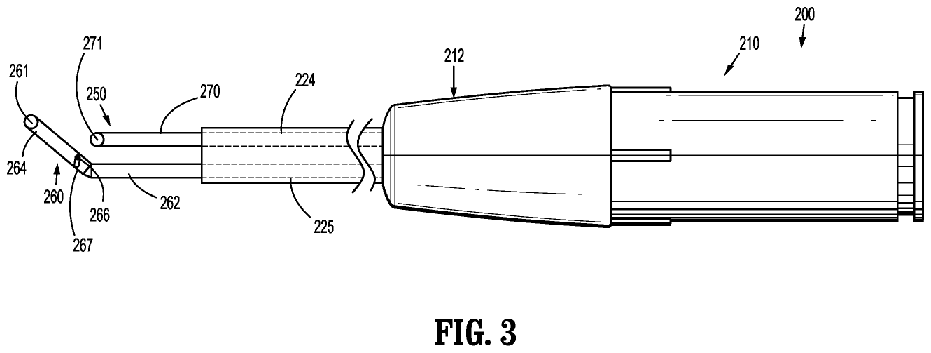

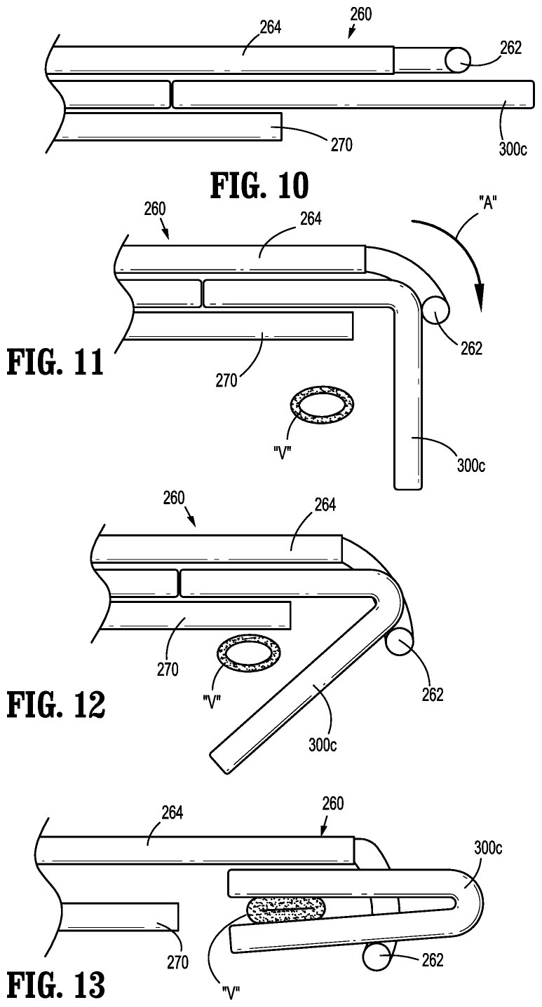

As illustrated in FIGS. 1-3, pair of jaw members 250 extends through elongated assembly 200, and includes first jaw member 260 and second jaw member 270. One or both jaw members 260, 270 may include a distal finger 261, 271, respectively, for engaging a portion of a surgical clip (e.g., 300a; see FIGS. 7-9). Distal fingers 261, 271 project laterally from the other portions of the respective jaw members 260, 270. First jaw member 260 includes distal portion 262 and proximal portion 264. Distal portion 262 is pivotable relative to proximal portion 264 through engagement between pin 266 and slot 267 (FIGS. 2 and 3; omitted from FIGS. 10-13 for clarity), in response to an additional or secondary translation of first slider 140. While not explicitly shown, the second jaw member 270 includes a similar orientation as first jaw member 260 with a distal portion that is pivotable relative to a proximal portion.

With particular reference to FIG. 4, surgical clip applier 10 retains one or a number of surgical clips 300 within outer shaft 222 for application to the desired tissue. The surgical clip applier 10 has an elongated clip channel member 302 for retaining a number of surgical clips 300 shown in an aligned manner above the clip channel member 302. The elongated clip channel member 302 is configured to remain longitudinally stationary relative to the elongated tubular member 14. The clip applier 10 includes a follower 306 connected to a follower spring 308. The follower spring 308 urges surgical clips 300 distally in the clip channel member 302. The clip applier 10 also includes a channel cover 310 that overlies the clip channel member 302 to retain and guide the follower 306 and the follower spring 308 and the clips 300 distally in the clip channel member 302. The clip applier 10 also includes a nose 312 to direct the clips 300 traversing through the clip channel member 302 into a channel defined between the jaw members 250.

With continued reference to FIG. 4, the surgical clip applier 10 includes a feed bar 400 for feeding clips 300 into the channel between the jaw members 250. The feed bar 400 includes a pusher spring 402 that biases the feed bar 400 in a longitudinal, i.e., distal, direction. The pusher spring 402 is disposed in a complementary location under a notch 404 defined in a trip block 406. The trip block 406 is adjacent to the clip channel cover member 304 on a distal side thereof. A hook 408 of the feed bar 400 engages the notch 404 of the trip block 406. The surgical clip applier 10 further includes a guide pin 401 disposed through the pusher spring 402 and that is configured to align the pusher spring 402. The hook 408 engages the guide pin 401 and the pusher spring 402 under the trip block 406. In this manner, the hook 408 is disposed through the notch 404 to engage the guide pin 401. The pusher spring 402 and guide pin 401 bias the feed bar 400 and permit the feed bar 400 to advance distally. Moreover, the guide pin 401 being disposed through the pusher spring 402 allows for a self-contained assembly. In order for spindle 128 to advance the pusher 400, the spindle 128 has a trip lever 500 and a biasing spring 502. The trip lever 500 is engaged with the feed bar 400 to advance the surgical clips 300 distally into the channel between the jaw members 250.

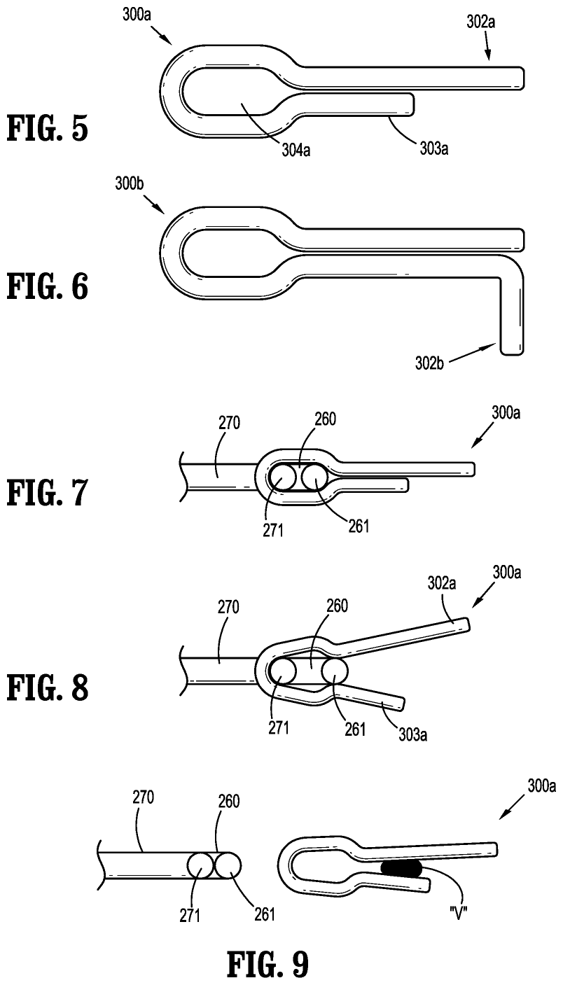

Referring to FIGS. 5-13, embodiments of clips and at various stages of deployment from surgical clip applier 10 are shown. With initial reference to FIGS. 5-9, clips are shown in the form of a first type of spring clip 300a (FIGS. 5 and 7-9) having an elongated leg or monopolar blade 302a, and a second type of spring clip 300b (FIG. 6) where one leg includes a bent distal end or a monopolar L-hook 302b. FIGS. 7-9 show spring clip 300a in various stages of deployment.

With continued reference to FIGS. 7-9, to deploy the clip 300a (e.g., onto a vessel “v”), distal fingers 261, 271 of first jaw member 260 and second jaw member 270, respectively, are positioned within an opening 304a (see FIG. 5) defined by clip 300a. Initially, in the position shown in FIG. 7, distal fingers 261 and 271 are in general close proximity to each other. Next, first jaw member 260 is translated distally relative to second jaw member 270 by distally translating first slider 140, for example. As shown in FIG. 8, this relative separation between distal fingers 261, 271 of respective jaw members 260, 270 causes legs 302a and 303a of clip 300a to move apart from each other, thereby opening the clip 300a. Then, the open clip 300a is moved toward a vessel “v”, and the first and second jaw member 260, 270 are approximated, which thereby closes the clip 300a onto the vessel “v” (FIG. 9). In this position, the distal fingers 261, 271 can be slid out from the opening 304a of the clip 300a, thus allowing the first and second jaw members 260, 270 to move proximally away from the closed clip 300a and vessel “v.” A subsequent clip 300a may then be loaded (e.g., automatically reloaded without user intervention) between distal fingers 261, 271 of respective jaw members 260, 270.

Referring now to FIGS. 10-13, another embodiment of the surgical clip applier 10 is shown. Here, pivoting of the distal portion 262 of first jaw member 260 relative to proximal portion 264 thereof is shown, and clips 300c are different from those discussed above with regard to FIGS. 5-9. Surgical clips 300c are advanced distally between first jaw member 260 and second jaw member 270. The surgical clips 300c are advanced in the same manner as discussed above with regard to surgical clip applier 10 of FIGS. 1-4.

Here, first jaw member 260 and second jaw member 270 are translatable longitudinally with respect to each other and independently of one another, via translation of first slider 140 and second slider 150, respectively. Further, first jaw member 260 includes distal portion 262 that is movable (e.g., pivotable) relative to proximal portion 264 thereof, in response to continued translation of first slider 140.

FIGS. 10-13 show a clip 300c at various stages of being deployed from between first jaw member 260 and second jaw member 270 of surgical clip applier 10. As discussed in further detail below, the clip 300c is able to be energized with electrosurgical energy to serve as a monopolar electrode. During surgery, such an energized clip 300c is usable to dissect and/or coagulate vasculature (e.g., relatively small vessels). Additionally, when a relatively large vessel is encountered during surgery, the same clip 300c that was used as a monopolar electrode can be deployed onto the vessel to ligate the vessel.

With continued reference to FIGS. 10-13, to deploy the clip 300c (e.g., onto a vessel “v”), the clip 300c and the first jaw member 260 are initially advanced distally (FIG. 10) relative to second jaw member 270. Next, the second jaw member 270 is advanced distally (FIG. 11) relative to first jaw member 260 such that second jaw member 270 is disposed between the vessel and the first jaw member 260 (e.g., portions of first jaw member 260, second jaw member 270, and vessel are longitudinally aligned). Next, the distal portion 262 of the first jaw member 260 is moved (e.g., pivoted) against the clip 300c and toward the second jaw member 270 (in the general direction of arrow “A” in FIG. 11) such that the clip 300c deforms (FIGS. 11 and 12). Finally, the second jaw member 270 is retracted proximally relative to the first jaw member 260, and distal portion 262 of the first jaw member 260 is further is moved (e.g., pivoted) against the clip 300c to further clamp clip 300c onto the vessel (FIG. 13).

The various clips 300, 300a, 300b, 300c of the disclosure are able to be energized with electrosurgical energy to serve as, for example, a monopolar electrode, although bipolar configurations, e.g., wherein the clip 300, 300a, 300b, 300c serves as one pole and an electrically-conductive portion of the clip applier 10 (that is isolated from the clip) serves as the other pole. There are various ways in which the clips 300-300c of surgical clip applier 10 can be energized. The clips 300-300c may be energized through direct contact with a conductive portion of the surgical clip applier 10 for example, such as the feed bar 400 that advances the clips 300-300c. The clips 300-300c may also be energized through a different conductive portion of surgical clip applier 10, 10a that makes direct or indirect contact with at least one clip 300-300c. In these embodiments, the conductive portion(s) of the surgical clip applier 10 may be connected to a lead wire that extends between the elongated assembly 200 and an actuation mechanism (e.g., a button 105 (FIG. 1)) on the handle assembly 100, or the conductive portion(s) can be part of a conductive path between elongated assembly 200 and the actuation mechanism of handle assembly 100. Further, the actuation mechanism is connected to or is configured to connect to a source of electrosurgical energy through a wire 130 (FIG. 1). Accordingly, selective engagement of the actuation mechanism transmits energy to the clips 300-300c.

In other embodiments, the clips 300-300c are energized by contacting a portion of the surgical clip applier 10 that is electrically coupled to a conductive portion thereof (that is in direct or indirect contact with at least one clip 300-300c) with an energized device (e.g., a monopolar pencil).

Thus, since the energizing of the clips 300-300c is performed selectively, energy can be supplied to the clips 300-300c to make the clips 300-300c function as a monopolar electrode before, during various stages of, or after actuation and/or formation of the clips 300-300c. For instance, the actuation mechanism may be configured to transmit energy to the distal-most surgical clip when the distal-most surgical clip engages (e.g., upon or substantially upon initial engagement with) tissue. In these embodiments, surgical clip applier 10 may include at least one sensor 170 that detects when tissue is engaged by a surgical clip (e.g., the distal-most surgical clip), for instance. While the sensor 170 is shown engaged with the end effector 250, the sensor 170 (and/or an additional sensor) may be engaged with a different portion of the clip applier 10, such that the handle assembly 100.

In embodiments, a distal-most of the clips 300-300c may be advanced to an extended, pre-formation position, e.g., as shown in FIG. 10, wherein a portion of the clip 300-300c defines a distally-extending linear electrode that can be selectively energized to treat tissue. In other embodiments, in the extended pre-formation position, the electrode portion of the clip 300-300c may define another suitable configuration, e.g., a hook-shaped configuration such as shown with respect to clip 300b in FIG. 6.

In additional or alternative embodiments, a distal-most one of the clips 300-300c may be partially formed such that the clip 300-300c is deformed to define a different-shaped electrode such as, for example, the hook-shaped electrode configurations of clip 300c illustrated in FIGS. 11 and 12 or the angled electrode configuration of clip 300a illustrated in FIG. 8. In any of these configurations, the partially-formed clip 300-300c may be selectively energized to treat tissue with the electrode portion thereof.

In either of the above configurations, once energy-based tissue treatment, e.g., using the electrode portion of the clip 300-300c in a pre-deployed or partially-deployed position or positions, is complete, the clip 300-300c may be fully formed about a vessel to clamp and close the vessel (see, e.g., FIGS. 12-13). Thus, this disclosure provides a device wherein a clip may be used as an energy-delivery electrode in one or more different configurations and then may be deployed for use as a traditional surgical clip (although additional or alternative energy application after or during formation is also contemplated).

Surgical instruments such as the surgical clip appliers described herein may also be configured to work with robotic surgical systems and what is commonly referred to as “Telesurgery.” Such systems employ various robotic elements to assist the surgeon and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely control the instruments via the robotic surgical system. As can be appreciated, a highly skilled surgeon may perform multiple operations in multiple locations without leaving his/her remote console which can be both economically advantageous and a benefit to the patient or a series of patients.

The robotic arms of the surgical system are typically coupled to a pair of master handles by a controller. The handles can be moved by the surgeon to produce a corresponding movement of the working ends of any type of surgical instrument (e.g., end effectors, graspers, knifes, scissors, etc.) which may complement the use of one or more of the embodiments described herein. The movement of the master handles may be scaled so that the working ends have a corresponding movement that is different, smaller or larger, than the movement performed by the operating hands of the surgeon. The scale factor or gearing ratio may be adjustable so that the operator can control the resolution of the working ends of the surgical instrument(s).

The master handles may include various sensors to provide feedback to the surgeon relating to various tissue parameters or conditions, e.g., tissue resistance due to manipulation, cutting or otherwise treating, pressure by the instrument onto the tissue, tissue temperature, tissue impedance, etc. As can be appreciated, such sensors provide the surgeon with enhanced tactile feedback simulating actual operating conditions. The master handles may also include a variety of different actuators for delicate tissue manipulation or treatment further enhancing the surgeon's ability to mimic actual operating conditions.

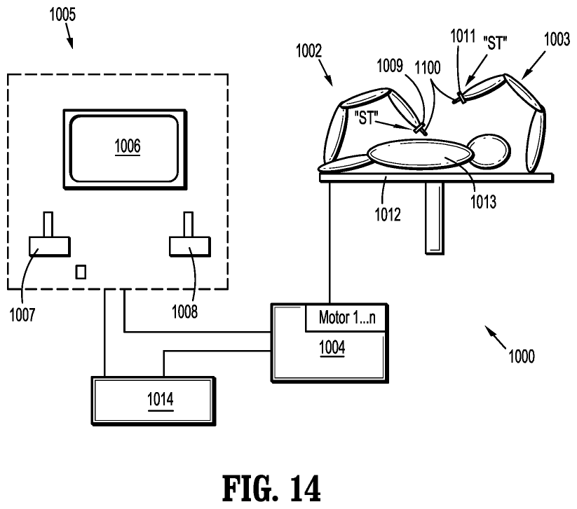

Referring to FIG. 14, a medical work station is shown generally as work station 1000 and generally may include a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a person (not shown), for example a surgeon, may be able to telemanipulate robot arms 1002, 1003 in a first operating mode.

Each of the robot arms 1002, 1003 may include a plurality of members, which are connected through joints, and an attaching device 1009, 1011, to which may be attached, for example, a surgical tool “ST” supporting an end effector 1100, in accordance with any one of several embodiments disclosed herein, as will be described in greater detail below.

Robot arms 1002, 1003 may be driven by electric drives (not shown) that are connected to control device 1004. Control device 1004 (e.g., a computer) may be set up to activate the drives, in particular by means of a computer program, in such a way that robot arms 1002, 1003, their attaching devices 1009, 1011 and thus the surgical tool (including end effector 1100) execute a desired movement according to a movement defined by means of manual input devices 1007, 1008. Control device 1004 may also be set up in such a way that it regulates the movement of robot arms 1002, 1003 and/or of the drives.

Medical work station 1000 may be configured for use on a patient 1013 lying on a patient table 1012 to be treated in a minimally invasive manner by means of end effector 1100. Medical work station 1000 may also include more than two robot arms 1002, 1003, the additional robot arms likewise being connected to control device 1004 and being telemanipulatable by means of operating console 1005. A medical instrument or surgical tool (including an end effector 1100) may also be attached to the additional robot arm. Medical work station 1000 may include a database 1014, in particular coupled to with control device 1004, in which are stored, for example, pre-operative data from patient/living being 1013 and/or anatomical atlases.

Accordingly, various endoscopic assemblies, constructed in accordance with the principles of the disclosure, may be provided which are also capable of firing or forming or closing surgical clips of various sizes, materials, and configurations, across multiple platforms for multiple different manufactures. For example, while the configuration of jaw members 250a and 250b have been shown and described for use with a surgical clip applier, it is contemplated and within the scope of the disclosure that the configuration of jaw members 250a, 250b may be incorporated into other surgical instruments, such as, for example, and not limited to, surgical staplers, tack appliers, and the like.

It should be understood that the foregoing description is only illustrative of the disclosure. Various alternatives and modifications can be devised by those skilled in the art without departing from the disclosure. Accordingly, the disclosure is intended to embrace all such alternatives, modifications and variances. The embodiments described with reference to the attached drawing figures are presented only to demonstrate certain examples of the disclosure. Other elements, steps, methods and techniques that are insubstantially different from those described above and/or in the appended claims are also intended to be within the scope of the disclosure.

What is claimed is:

1. A clip applier, comprising:
a handle;
an elongated shaft extending distally from the handle;
an end effector operably engaged to a distal portion of the elongated shaft including a longitudinal axis defined therethrough, the end effector including a first jaw member and a second jaw member;
at least one surgical clip disposed within the elongated shaft and selectively loadable between the first and second jaw members;
a trigger configured to selectively translate at least a portion of the first jaw member relative to the second jaw member upon actuation thereof in a direction parallel to the longitudinal axis; and
an actuation mechanism adapted to connect to a source of electrosurgical energy and configured to selectively transmit energy to at least one of the at least one surgical clip.

2. The clip applier according to claim 1, wherein a distal portion of the first jaw member is movable relative to the second jaw member and relative to a proximal portion of the first jaw member to deform a surgical clip of the at least one surgical clip.

3. The clip applier according to claim 1, wherein the actuation mechanism is configured to transmit energy to a distal-most surgical clip of the at least one surgical clip.

4. The clip applier according to claim 3, wherein the actuation mechanism is configured to transmit energy to the distal-most surgical clip during formation of the distal-most surgical clip.

5. The clip applier according to claim 3, wherein the actuation mechanism is configured to transmit energy to the distal-most surgical clip while the first jaw member is stationary relative to the second jaw member.

6. The clip applier according to claim 3, wherein the actuation mechanism is configured to transmit energy to the distal-most surgical clip while at least a portion of the first jaw member is longitudinally translating relative to the second jaw member via actuation of the trigger.

7. The clip applier according to claim 3, wherein the actuation mechanism is configured to transmit energy to the distal-most surgical clip while at least a portion of the first jaw member is distally translating relative to the second jaw member via actuation of the trigger.

8. The clip applier according to claim 3, wherein the actuation mechanism is configured to transmit energy to the distal-most surgical clip when the distal-most surgical clip is engaging tissue.

9. The clip applier according to claim 1, wherein actuation of the trigger is configured to cause at least a portion of the first jaw member to move relative to the second jaw member in a direction that is at a non-parallel angle relative to the longitudinal axis.

10. The clip applier according to claim 1, wherein actuation of the trigger is configured to cause translation of the second jaw member relative to the elongated shaft in a direction that is parallel to the longitudinal axis.

11. A method of treating tissue, comprising:
translating at least a portion of a first jaw member of a clip applier relative to a second jaw member of the clip applier, while simultaneously moving a surgical clip from a first position where the surgical clip is in contact with the first jaw member and the second jaw member, to a second position where the surgical clip engages tissue and dislodges from the first and second jaw members; and
energizing the surgical clip.

12. The method according to claim 11, further comprising automatically moving a second surgical clip from a position proximally of the first position into the first position.

13. The method according to claim 11, wherein translating at least a portion of the first jaw member relative to the second jaw member causes formation of the surgical clip.

14. The method according to claim 13, wherein energizing the surgical clip is performed during formation of the surgical clip.

15. The method according to claim 11, wherein energizing the surgical clip is performed while the first jaw member is stationary relative to the second jaw member.

16. The method according to claim 11, wherein energizing the surgical clip is performed while at least a portion of the first jaw member is longitudinally translating relative to the second jaw member.

17. The method according to claim 11, wherein energizing the surgical clip is performed while the surgical clip is engaging tissue.

18. The method according to claim 11, further comprising moving at least a portion of the first jaw member relative to the second jaw member in a direction that is non-parallel relative to the longitudinal axis.
